(19) United States
(12) Patent Application Publication
(10) Pub. No.: US 2019/0269858 A1
(43) Pub. Date: Sep. 5, 2019

(54) MULTI-CHANNEL ROTARY ENCODER

(71) Applicant: Verily Life Sciences LLC, South San Francisco, CA (US)

(72) Inventor: Adam Reich, Oakland, CA (US)

(21) Appl. No.: 16/284,962

(22) Filed: Feb. 25, 2019

Related U.S. Application Data
(60) Provisional application No. 62/638,664, filed on Mar. 5, 2018.

(51) Int. Cl. A61M 5/315; G01D 5/252

(52) U.S. Cl. CPC A61M 5/31551; G01D 5/252; A61M 5/31528; A61M 5/31568; A61M 5/31585

(57) ABSTRACT

A rotary encoder includes a substrate, two or more switches disposed on the substrate, a mechanical wave generator, and a controller. The mechanical wave generator is disposed proximate to the substrate. The substrate and the mechanical wave generator are adapted to rotate relative to each other about a central axis. The mechanical wave generator has a profile shape that repeats circumferentially about the central axis. The profile shape has ridges and valleys that engage the two or more switches to activate and deactivate the two or more switches as the substrate and the mechanical wave generator rotate relative to each other. The controller is electrically coupled to the two or more switches to track activations of the two or more switches and digitally encode a rotational position of the substrate relative to the wave generator based upon the activations.

### CROSS-REFERENCE TO RELATED APPLICATION

This application claims the benefit of U.S. Application No. 62/638,664, filed on Mar. 5, 2018, the contents of which are incorporated herein by reference.

### TECHNICAL FIELD

This disclosure relates to rotary encoders, and in particular but not exclusively, relates to dosage-tracking of a drug injection pen using a rotary encoder.

### BACKGROUND INFORMATION

A rotary encoder is a device that converts an angular position or rotational motion of a shaft to a signal, which may be used to track the angular position or rotational motion of the shaft. Rotary encoders can be classified into two subcategories: absolute rotary encoders and relative rotary encoders. Absolute rotary encoders identify the absolute angular position of the shaft at a given moment while relative rotary encoders identify the motion of the shaft, which can be tracked to calculate the absolute angular position relative to a starting position. A relative rotary encoder may use an extraneous counter to maintain state information in order to compute the absolute angular position of the shaft.

Rotary encoders often use multiple “tracks” to increase the resolution for encoding angular position or rotational motion of the shaft. Tracks are often implemented as a “ring pattern” on the shaft.

### BRIEF DESCRIPTION OF THE DRAWINGS

Non-limiting and non-exhaustive embodiments of the invention are described with reference to the following figures, wherein like reference numerals refer to like parts throughout the various views unless otherwise specified. Not all instances of an element are necessarily labeled so as not to clutter the drawings where appropriate. The drawings are not necessarily to scale, emphasis instead being placed upon illustrating the principles being described.

FIG. 1 illustrates a drug injection system, in accordance with an embodiment of the disclosure.

FIG. 2A illustrates part of an injection pen and a pen button, including a dosage measurement system that uses a rotary encoder, in accordance with an embodiment of the disclosure.

FIG. 2B illustrates a partial cross section of the pen button and injection pen of FIG. 2A, in accordance with an embodiment of the disclosure.

FIG. 2C illustrates the pen button of FIG. 2A inserted into the pen body, in accordance with an embodiment of the disclosure.

FIG. 2D illustrates a partial cross section of the pen button and injection pen of FIG. 2C, in accordance with an embodiment of the disclosure.

FIG. 2E illustrates an exploded view of the pen button including the rotary encoder, in accordance with an embodiment of the disclosure.

FIG. 3 illustrates a substrate with two electro-mechanical switches and a controller disposed thereon, which are components of a rotary encoder, in accordance with an embodiment of the disclosure.

FIG. 4 illustrates a mechanical wave generator of the rotary encoder, in accordance with an embodiment of the disclosure.

FIGS. 5A-5E illustrate various profile shapes of the mechanical wave generator, in accordance with embodiments of the disclosure.

FIG. 6 is a flow chart illustrating operation of a rotary encoder disposed within a drug injection pen, in accordance with an embodiment of the disclosure.

### DETAILED DESCRIPTION

Embodiments of a system, apparatus, and method of operation for a multi-channel rotary encoder are described herein. In the following description numerous specific details are set forth to provide a thorough understanding of the embodiments. One skilled in the relevant art will recognize, however, that the techniques described herein can be practiced without one or more of the specific details, or with other methods, components, materials, etc. In other instances, well-known structures, materials, or operations are not shown or described in detail to avoid obscuring certain aspects.

Reference throughout this specification to “one embodiment” or “an embodiment” means that a particular feature, structure, or characteristic described in connection with the embodiment is included in at least one embodiment of the present invention. Thus, the appearances of the phrases “in one embodiment” or “in an embodiment” in various places throughout this specification are not necessarily all referring to the same embodiment. Furthermore, the particular features, structures, or characteristics may be combined in any suitable manner in one or more embodiments.

Measuring the quantity and recording the timing of a drug's administration is an integral part of many disease treatments. For many treatments, to achieve the best therapeutic effect, specific quantities of a drug may need to be injected at specific times of day. For example, individuals suffering from diabetes may be required to inject themselves regularly throughout the day in response to measurements of their blood glucose. The frequency and volume of insulin injections should be carefully tracked and controlled to keep the patient's blood glucose level within a healthy range.

Currently, there are a limited number of methods or devices capable of tracking drug administration without requiring the user to manually measure and record the volume, date, and time. A variety of glucose injection syringes/pens have been developed, but there is much room for significant advancement in the technology in order to reduce the size, lower the cost, enhance the functionality, and improve the accuracy. Thus, the current technology may not be an ideal long-term solution. For example, current insulin pens are often disposable, but do not include dosage tracking functionality. A smaller portion of the market is composed of reusable pens which are more expensive. Embodiments of the rotary encoder descried herein are well suited for accurately tracking dispensed dosages of a drug from an injectable pen.

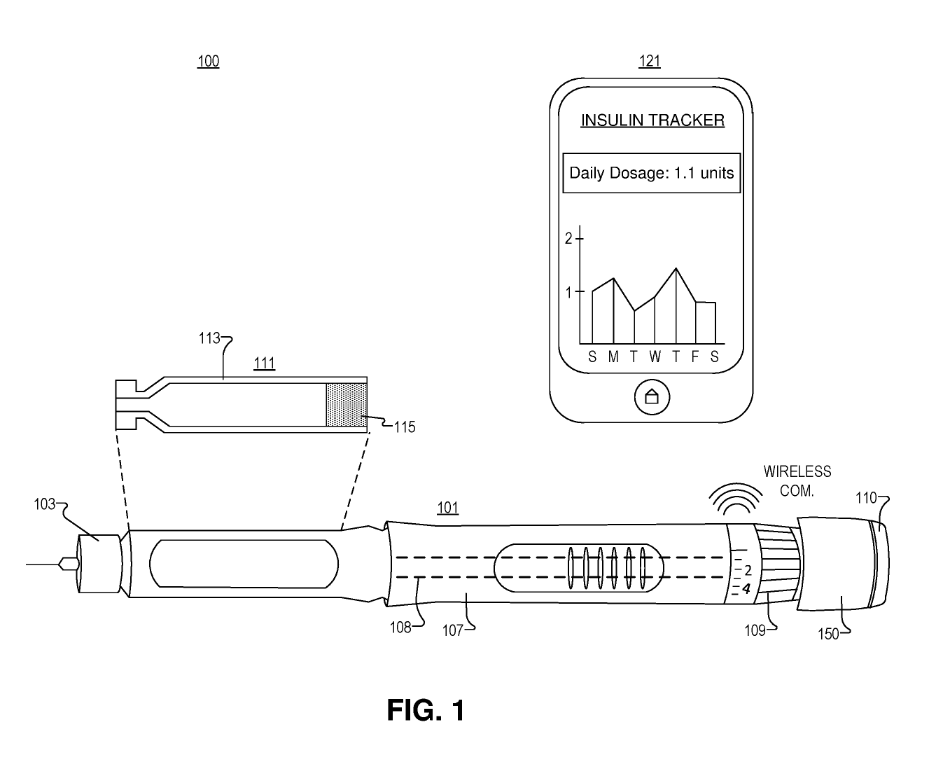

FIG. 1 illustrates a drug injection system 100, in accordance with an embodiment of the disclosure. Drug injection system 100 includes injection pen 101, drug cartridge 111, and processing device 121 (e.g., a portable computing device, a smart phone, etc.).

Drug cartridge 111 includes cartridge body 113 and plunger head 115. In the depicted embodiment, plunger head 115 starts near the rear of drug cartridge 111 and is pushed forward in drug cartridge 111 by a leadscrew 108 of a dosage injection mechanism disposed in injection pen 101. This forces medication/fluid out of the narrow end of drug cartridge 111 when a user chooses to dispense a fluid. In one embodiment, cartridge body 113 includes borosilicate glass.

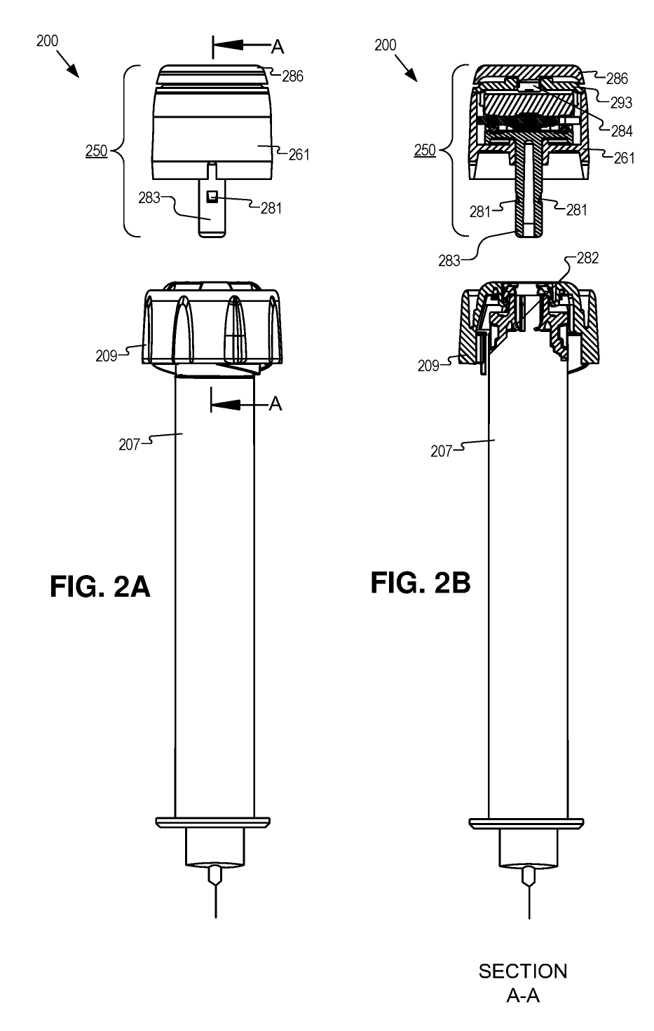

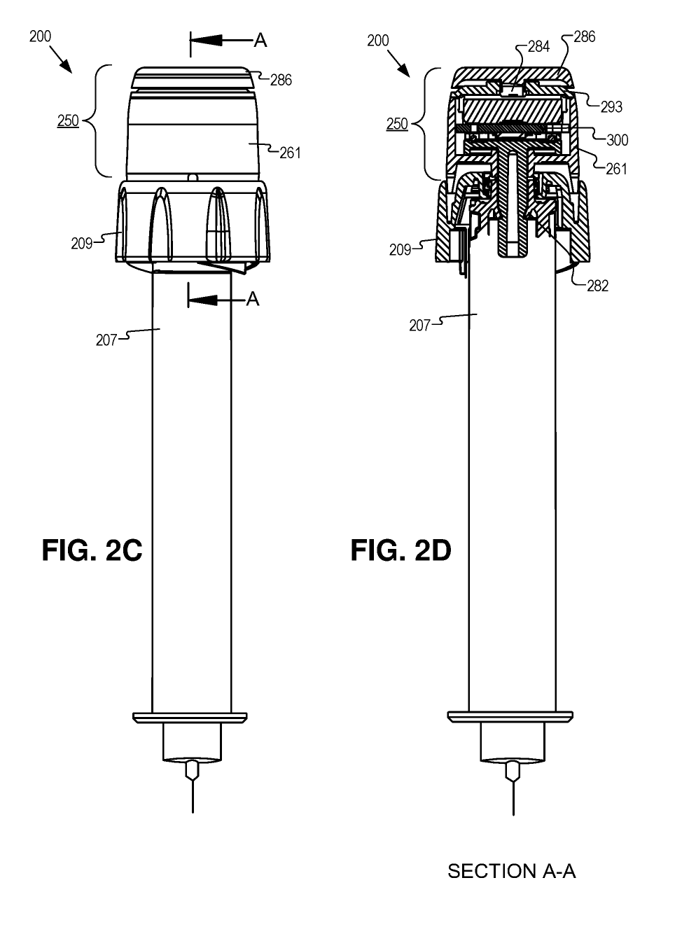

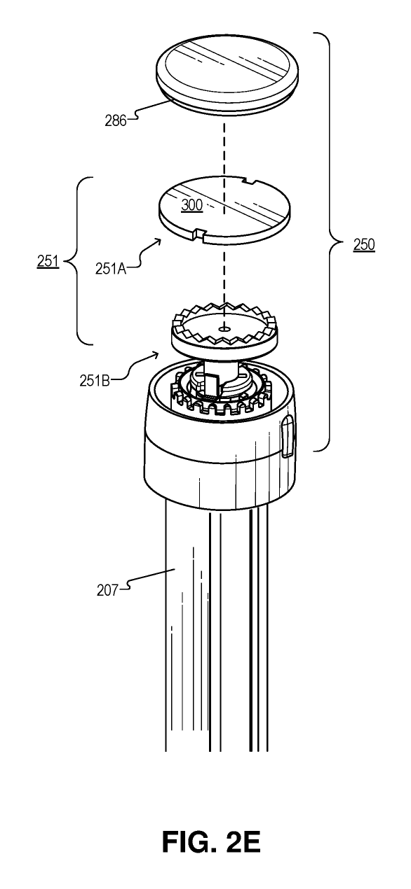

Injection pen 101 is a hand-held device and includes needle 103, body/housing 107 (including a dosage injection mechanism to push in plunger head 115 and expel fluid from drug cartridge 111), drug delivery control wheel 109 (twist wheel or dial to “click” select the dosage), and pen button 150 (which includes push button 110 to dispense the selected quantity of the fluid from cartridge 111). In one embodiment, pen button 150 includes a dosage measurement system (see e.g., FIGS. 2A-2E). As shown, housing 107 is configured to accept cartridge 111. In one embodiment, cartridge 111 may be disposed in an insert which screws/snaps onto the bulk of housing 107. However, as one of ordinary skill in the art will appreciate, injection pen 101 can assume other configurations and have other components.

As stated, injection pen 101 includes a housing/body 107 shaped to accept a cartridge containing a fluid, and also includes a dosage injection mechanism positioned in the housing 107 to produce a rotational motion that advances a leadscrew against plunger head 115 and forces the fluid out of the cartridge when the drug injection pen 101 dispenses the fluid. A dosage measurement system is also disposed in the pen (e.g., in button 150 or elsewhere in pen body 107) to track the rotational motion of the dosage injection mechanism. The dosage measurement system encodes the rotational motion of the dosage injection mechanism to track the amount of fluid dispensed and further outputs a signal indicative of the rotation or fluid dispensed.

A controller is also disposed in drug injection pen 101, as part of the dosage measurement system. The controller includes logic that when executed by the controller causes the controller to record the electrical signals indicative of the fluid dispensed into a dispensing log. One of ordinary skill in the art will appreciate that the controller may be static (e.g., have logic in hardware), or dynamic (e.g., have programmable memory that can receive updates). In some embodiments, the controller may register the electrical signal output from the dosage measurement system as an injection event of the fluid, and the controller may calculate a quantity of the fluid dispensed based, at least in part, on a number of the injection events of the fluid registered by the controller. It is appreciated that this circuitry, which will be described in greater detail in connection with other figures, may be disposed anywhere in drug injection pen 101 (e.g., in body/housing 107 or pen button 150), and in some instances, logic may be distributed across multiple devices.

Processing device 121 (e.g., a smartphone, tablet, general purpose computer, distributed system, servers connect to the internet, or the like) may be coupled to receive dosage data from injection pen 101 to store/analyze this data. For instance, in the depicted embodiment, processing device 221 is a smartphone, and the smartphone has an application that records how much insulin has been dispensed from injection pen 101. In the illustrated embodiment, the application plots how much insulin has been injected by the user over a historical period of time (e.g., week). In this embodiment, a power source is electrically coupled to the controller in injection pen 101, and a transceiver is electrically coupled to the controller to send and receive data to/from processing device 121. Here, data includes information indicative of a quantity of the fluid dispensed. Transceiver may include Bluetooth, RFID, or other wireless communications technologies.

FIG. 2A illustrates a pen housing 207 and pen button 250 of a drug injection pen 200, in accordance with an embodiment of the disclosure. Pen button 250 includes a dosage measurement system. Drug injection pen 200 is one possible implementation of injection pen 101 illustrated in FIG. 1. As shown, pen button 250 is fabricated to be inserted into the back end of injection pen 200 (opposite the dispensing end). Pen button 250 includes a pair of notches 281, cut into a shaft/column 283 protruding from pen button 250, which clip into pen housing 207. In the illustrated embodiment, the pen button housing 261 contains the dosage measurement system including electronics to measure a rotational motion of the dosage injection mechanism that is related to a volume of fluid dispensed.

FIG. 2B illustrates a partial cross section of relevant portions of drug injection pen 200 including pen button 250 and pen housing 207, in accordance with an embodiment of the disclosure. As depicted, the pair of notches 281 is cut into shaft 283 protruding from pen button 250. A pair of locking tabs 282 are disposed in the pen housing 107 that fit into notches 281, and provide both axial restraint (so pen button 250 doesn't fall out), and also connects the dosage injection mechanism to the dosage measurement system. The body of pen button 250 is rotationally locked to the drug delivery control wheel (the largest diameter part in FIG. 2B) via two slots.

FIG. 2C illustrates pen button 250 of FIG. 2A inserted into pen body 207, in accordance with an embodiment of the disclosure. As shown pen button 250 clips into the back end of the injection pen, so that the drug delivery control wheel 209 is disposed between pen button 250 and pen housing 207. Accordingly, in the illustrated embodiment, a component in the dosage measurement system clips to the dosage injection mechanism in the drug injection pen.

FIG. 2D illustrates a partial cross section of relevant portions of the pen button 250 and the fully assembled injection pen 200 of FIG. 2C, in accordance with an embodiment of the disclosure. As shown, the pair of locking tabs 282 fit into notches 281 to hold pen button 250 in place. In some embodiments, pen button 250 can be fabricated separately from the rest of the injection pen and then “snapped” into pen housing 207. Thus, the pen assembly process merely involves rotational alignment of the button 250 notches with the pins in the drug delivery control wheel 209, and alignment of the notches 281 in the button shaft 283 to locking tabs 282. Then, pen button 250 is pressed straight into the pen. Locking tabs 282 are tapered so that they allow insertion, but not removal.

An additional aspect of the embodiment depicted is that pen button 250 spins when the pen dispenses fluid. In the depicted embodiment, pen button 250 rotates along with drug delivery control wheel 209 when the pen is dispensing a dose. So that the user's thumb does not interfere with this rotation, a thrust bearing 284 and spinner 286 are disposed on a top side of pen button 250. Thus components of pen button 250 spin when the injection pen dispenses fluid, but the user's thumb and fingers do not prevent dispensing of the fluid. In other words, a first portion of the button housing (e.g., the sides of the button housing 261) is coupled to rotate around a longitudinal axis of the drug injection pen when attached to the dosage injection mechanism, and a second portion of the button housing (e.g., spinner 286) is coupled to rotate independently from the first portion.

In some embodiments, spinner 286 may be made from polybutylene terephthalate (e.g., Celanex 2404MT). Spinner 286 may interact mechanically with (and bear on) button housing 261, and housing clip 293. Housing clip 293 may be made from polycarbonate (e.g., Makrolon 2458). In the illustrated embodiment, housing clip 293 snap fits to housing 261, and spinner 286 bears directly on housing clip 293. A component portion 251b (e.g., also referred to as a waveform generator) may also be made from polycarbonate, and snaps into a clutch in the pen. Component 251b may also bear on housing 261. Housing 261 may be made from polyoxymethylene (e.g., Hostaform MT8F01). Housing 261 may bear on a clutch within the drug injection pen, spinner 286, and a linear slide track on the drug delivery control wheel 209. Drug delivery control wheel 209 may also be made from polycarbonate, and interacts with the linear slide track on housing 261.

In operation, the components may move together according to the following steps (discussed from a user-fixed reference frame). A user may dial a dose using drug delivery control wheel 209. The user presses down on spinner 286 with their thumb. Spinner 286 presses housing 261 down. Housing 261 presses the clutch inside the pen body down, and the clutch disengages. Drug delivery control wheel 209 and housing 261 will spin with a substrate 300 as the drugs are dispensed and component portion 251b/spinner 286 stay rotationally stationary. Thus, drug delivery control wheel 209, housing 261, and substrate 300 are mechanically coupled to rotate when fluid is dispensed. Tabs on substrate 300 interact with features on the inside of housing 261 to spin substrate 300. It is noteworthy that while dialing a dose, there may be no relative motion between component portion 251b and substrate 300, but while dispensing, substrate 300 rotates relative to component portion 251b, which is fixed to the user-reference frame. In other embodiments, the relative motion may occur while dialing in a dose prior to actually dispensing the fluid. In such embodiments, the delivery control wheel or dial grip may be considered part of the dosage injection mechanism.

In some embodiments, component portion 251b is connected to the clutch (contained in the pen body and included in the dosage injection mechanism)—these parts may not move relative to one another. The clutch is connected to the drive sleeve (also included in the dosage injection mechanism)—which moves axially relative to the clutch with about 1 mm range of motion. The leadscrew is threaded into the drive sleeve. If the user has dialed a dose and applies force to button 250, the clutch releases from the sleeve and a leadscrew is pushed through a threaded “nut” in the pen body causing the leadscrew to advance. When the leadscrew advances, it presses on the plunger head in the medication vial to dispense fluid. It should be appreciated that the instant application is not intended to be limited to any particular dosage injection mechanism, but rather is intended to be broadly applicable to a variety of dosage injection mechanisms that generate a rotational motion.

FIG. 2E illustrates an exploded view of pen button 250 of FIG. 2A, in accordance with an embodiment of the disclosure. As shown, pen button 250 includes a number of components of the dosage measurement system that are stacked in a layered configuration in the pen button 250. The dosage measurement system tracks a delivered dosage of a drug by tracking the rotational motion of internal components using a multi-channel rotary encoder 251. Rotary encoder 251 tracks the relative rotation of internal components of drug injection pen 200, which correlates to the delivered dosage of a fluid. Rotary encoder 251 is described in connection with drug injection pen 200; however, it should be appreciated that rotary encoder 251 may be used with a variety of different types of drug injection pens and furthermore, may be implemented in any device where it is desired to track or measure a rotational motion between two components.

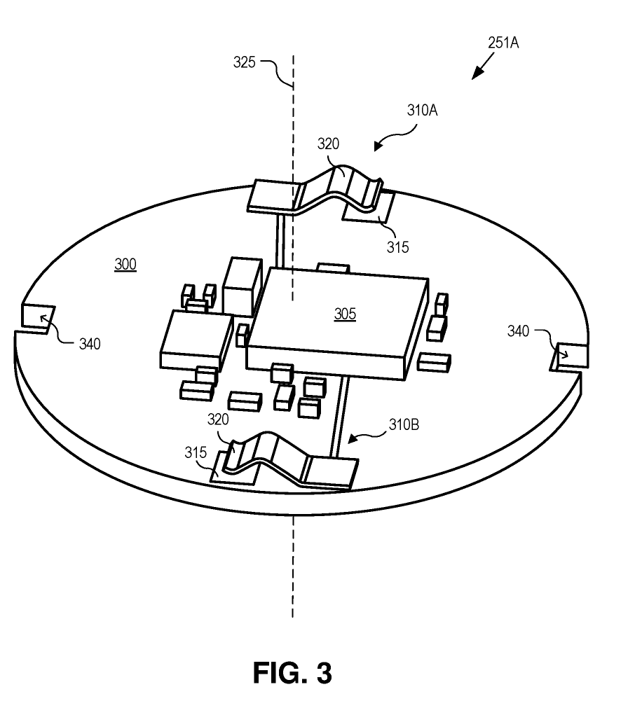

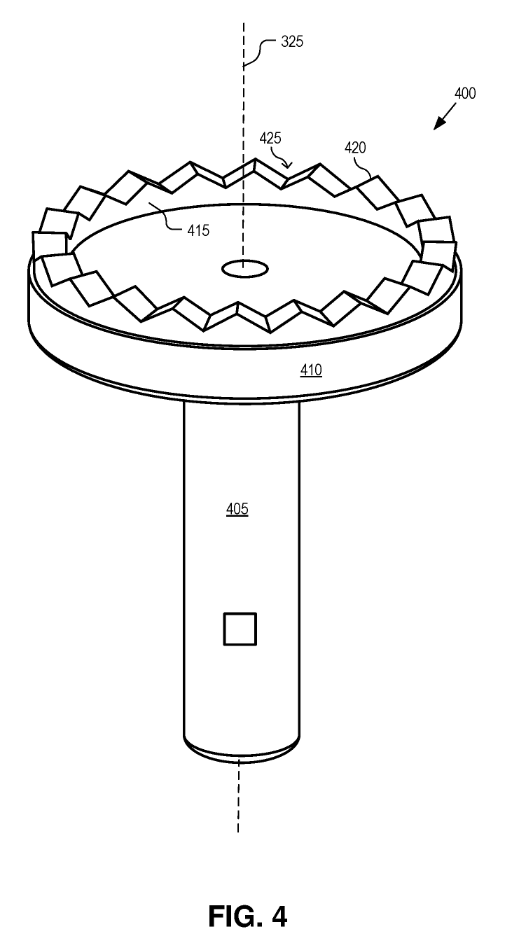

The illustrated embodiment of rotary encoder 251 includes first and second portions 251A and 251B, respectively, which rotate relative to each other. In the illustrated embodiment, many of the sub-components of first portion 251A are disposed on the side of substrate 300 that faces second portion 251B and are thus out of view in the illustration of FIG. 2E. FIG. 3 illustrates the first portion 251A from its other side. The illustrated embodiment of first portion 251A includes a circuit board or substrate 300 having a controller 305 and two electro-mechanical switches 310A and 310B (collectively referred to as 310) disposed thereon. It should be appreciated that various other embodiments of rotary encoder 251 may include only a single electro-mechanical switch 310 or more than two electro-mechanical switches 310. Other components, such as memory, wireless transmitter/transceiver, etc., may also be disposed on substrate 300. Each switch 310 includes a contact pad 315 and flexible contact lead 320. The second portion 251B includes a mechanical wave generator 400 (see FIG. 4). The illustrated embodiment of mechanical wave generator 400 (corresponding to second portion 251B) includes a shaft 405, a platform 410, and a ring substrate 415 having a profile shape with ridges 420 and valleys 425 that repeat.

When substrate 300 (first portion 251A) and mechanical wave generator 400 (second portion 251B) are assembled into rotary encoder 251, ridges 420 are co-radially aligned about a central axis 325 with flexible contact leads 320. During operation, substrate 300 and mechanical wave generator 400 rotate relative to each other about central axis 325. Ridges 420 are positioned relative to flexible contact leads 320 to pass over switches 310 and with incremental changes in the relative angular position of substrate 300 to mechanical wave generator 400, flexible contact leads 320 are reciprocally pushed against contact pads 315 to form closed and open circuits, which activate and deactivate switches 310. Controller 305 is electrically coupled to switches 310 to track each activation/deactivation of switches 310 and digitally encode a rotational position of substrate 300 relative to mechanical wave generator 400.

In the illustrated embodiment, controller 305 is disposed directly on substrate 300. In this embodiment, electrical traces may extend from controller 305 to switches 310 along substrate 300. In other embodiments, controller 305 may be remotely disposed off of substrate 300 and otherwise electrically connected to switches 310. In one embodiment, controller 305 may be implemented with a microcontroller coupled to memory that stores instructions that are executed by the microcontroller. In another embodiment, controller 305 is implemented using hardware logic, such as an application specific integrated circuit (ASIC), a field programmable gate array (FPGA), or otherwise. A combination of both hardware logic and software logic/instructions may also be used to implement controller 305.

In the illustrated embodiment, electro-mechanical switches 310 are co-radially aligned on substrate 300. Each electro-mechanical switch 310 is angularly offset from each other on substrate 300 such that only a single one of the electro-mechanical switches 310 is activated at a given time by ridges 420. Accordingly, each switch 310 corresponds to an encoding channel of rotary encoder 251. The total number of encoding states per revolution between substrate 300 and mechanical wave generator 400 is twice the total number of ridges 420 times a total number of electro-mechanical switches 310. Although FIGS. 3 and 4 illustrate two electro-mechanical switches 310 and twenty ridges 420 for a total number of 40 encoding states per revolution, other embodiments may include less or more electro-mechanical switches 310 and/or less or more ridges 420.

The illustrated embodiments of switches 310 each include a contact pad 315 and flexible contact lead 320. Flexible contact leads 320 each have a first end mounted to substrate 300 and a second end that is cantilevered over contact pad 315 such that it can be pushed by ridges 420 against contact pad 315. In one embodiment, ridges 420 of ring substrate 415 are fabricated of electrically insulating material (e.g., plastic) while flexible contact leads 320 and contact pads 315 are fabricated of electrically conductive materials (e.g., metal). Of course, it should be appreciated that other configurations of electro-mechanical switches may be implemented. Furthermore, it is anticipated that electro-mechanical switches 310 may be mounted to substrate 300 in other manners and in other positions than illustrated. Furthermore, in other embodiments electro-mechanical switches 310 may be implemented using other types of switching mechanisms, such as magnetic switches, etc.

Mechanical wave generator 400 is referred to as a “wave generator” due to the profile shape of the ridges 420 and valleys 425 of ring substrate 415. The profile shape repeats circumferentially (or angularly) about central axis 325. As substrate 300 and mechanical wave generator 400 rotate relative to each other, the profile shape activates and deactivates electro-mechanical switches 310 according to the waveform generated by the profile shape. Shaft 405 may be coupled to other components.

In one embodiment, substrate 300 is stationary and mechanical wave generator 400 is mounted to rotate. In one embodiment, shaft 405 operates as a linkage to other components of a larger system (e.g., a dosage injection mechanism of drug injection pen 200), which drives shaft 405 with a rotational torque about central axis 325. Since shaft 405 is mounted to platform 410 upon which ring substrate 415 is disposed, the rotation of shaft 405 is encoded by controller 305. In another embodiment, mechanical wave generator 400 is rotationally stationary and substrate 300 is mounted to rotate (e.g., via cutouts 340) relative to mechanical wave generator 400. In yet another embodiment, both mechanical wave generator 400 and substrate 300 are mounted to rotate.

Rotary encoder 251 includes fewer separable components and fewer components moving relative to each other when compared to conventional rotary encoders that include a separate wiper contact. Fewer distinct components make for easier assembly and simpler electrical connections between the various components. For example, contact pads 315, flexible contact leads 320, and controller 305 are all disposed on a common substrate 300 which permits fixed trace lines for establishing electrical connections.

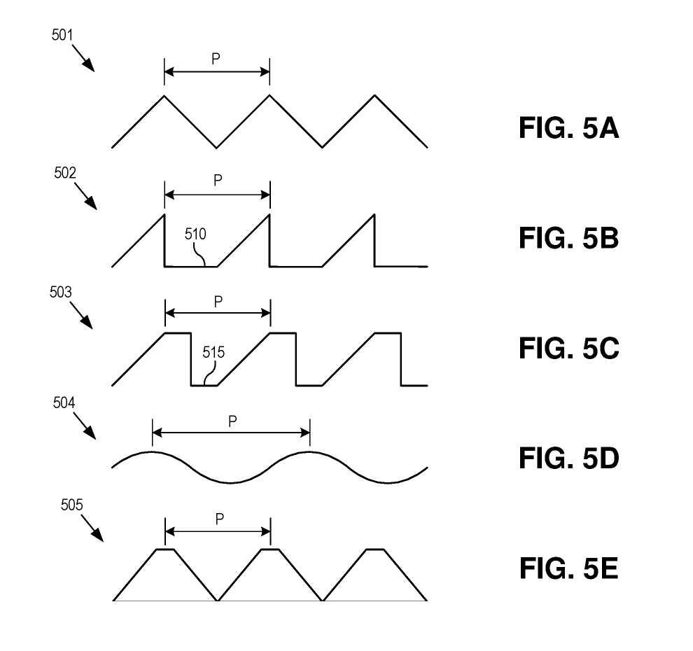

It should be appreciated that the profile shape of ring substrate 415 may assume a variety of different shapes. Referring to FIGS. 5A-5E, a variety of different profile shapes for ring substrate 415 are illustrated. For example, FIGS. 5A and 5B illustrate demonstrative triangular shapes 501 and 502. FIG. 5A illustrates connected triangular shapes 501 while FIG. 5B illustrates triangular shapes 502 separated by gaps 510. FIG. 5C illustrates truncated triangular shapes 503 also separated by gaps 515. FIG. 5D illustrates an undulating shape 504. FIG. 5E illustrates trapezoidal shapes 505.

In each profile shape, the separation distance between ridges of each repeating shape corresponds to the pitch P of mechanical wave generator 400. The smaller the pitch P, the greater the number of encoding states per revolution of mechanical wave generator 400. However, the pitch P can be traded off with the number of electro-mechanical switches 310 on substrate 300 to achieve the same number of encoding states per revolution. Accordingly, increasing the number of electro-mechanical switches 310 can enable an increase in the pitch P to achieve the same encoder resolution. By increasing the pitch P, the frequency of each digital signal of a given channel encoded by controller 305 is reduced. This per channel frequency reduction simplifies debouncing of the digital signals and enables higher rotational speeds than may otherwise be possible.

In one embodiment, the profile shapes and relative spacing of electro-mechanical switches 310 are selected to achieve mechanically stable angular positions for each sequential activation of an electro-mechanical switch 310. For example, in the case of two electro-mechanical switches 310, the relative offset positions between ridges 420 may be an increment of half the pitch P. In the case of three electro-mechanical switches 310, the relative offset positions may be an increment of one third the pitch P. Other offset increments may be implemented. In other embodiments, the profile shape may be selected to reduce friction, increase longevity of switches 310, or otherwise.

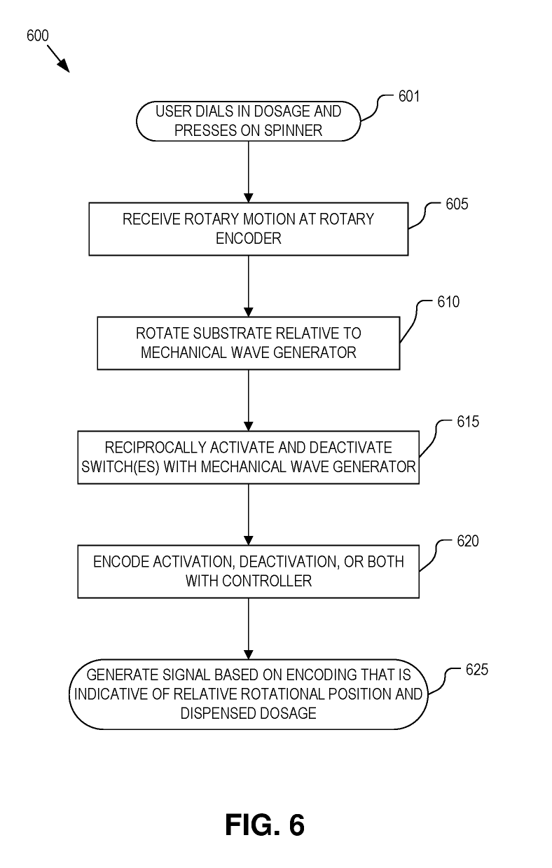

FIG. 6 is a flow chart illustrating a process 600 of operation of rotary encoder 251 disposed within drug injection pen 101 (or 200), in accordance with an embodiment of the disclosure. The order in which some or all of the process blocks appear in process 600 should not be deemed limiting. Rather, one of ordinary skill in the art having the benefit of the present disclosure will understand that some of the process blocks may be executed in a variety of orders not illustrated, or even in parallel.

In a process block 601, a user of drug injection pen 200 (or 101) dials in a desired dosage with control wheel 209, which represents an example dosage selection mechanism. Once the dosage is selected, the user presses with their thumb on spinner 286 located at the rear distal end of pen button 250. In one embodiment, the pressure from the user's thumb is translated into a rotary motion by the dosage injection mechanism of drug injection pen 200 while the fluid is being dispensed. In yet another embodiment, the rotary motion is derived from the user rotating control wheel 209 when they dial in the desired dosage.

In a process block 605, the rotary motion is received by rotary encoder 251. As mentioned above, the rotary motion may be received while the dosage is being dispensed (e.g., from the dosage injection mechanism) or prior to dispensing when the dosage is selected with control wheel 209 (e.g., from the dosage selection mechanism).

In a process block 610, the rotary motion causes substrate 300 to rotate relative to mechanical wave generator 400. In one embodiment, substrate 300 rotates while mechanical wave generator 400 remains rotationally fixed. In another embodiment, substrate 300 remains rotationally fixed while mechanical wave generator 400 rotates. In yet another embodiment, both substrate 300 and mechanical wave generator 400 both rotate in opposite directions or at different rates, thereby generating a relative rotation that is related to the dosage delivered.

In a process block 615, the relative rotation between substrate 300 and mechanical wave generator 400 causes ridges 420 to reciprocally engage flexible contact lead 320. This engagement in turn reciprocally presses flexible contact leads 320 against contact pads 315, resulting in a reciprocal activations and deactivations of switches 310.

In a process block 620, the reciprocal activations and deactivations are monitored by controller 305 and encoded into a stateful value that tracks the absolute rotational position of substrate 300 relative to mechanical wave generator 400. In one embodiment, just the activations are encoded. In another embodiment, just the deactivations are encoded. In yet another embodiment, both activations and deactivations are encoded.

Finally, in a process block 625, controller 305 generates a signal, based upon the encoding of the activations and/or deactivations of one or more switches 310, that is indicative of the dosage of a fluid disposed over a period of time. In one embodiment, the signal is a stateful value representative of the absolute rotational position between substrate 300 and mechanical wave generator 400 as measured from a zeroed position or reference position. This signal may be wirelessly transmitted from drug injection pen 101 to processing device 121 periodically or on-demand. It should be appreciated that process 600 represents one example use case scenario of rotary encoder 251 with a drug injection pen; however, rotary encoder 251 is well suited to encode rotary motions in a variety of other types of devices.

The processes explained above may be described in terms of computer software and hardware. The techniques described may constitute machine-executable instructions embodied within a tangible or non-transitory machine (e.g., computer) readable storage medium, that when executed by a machine will cause the machine to perform the operations described. Additionally, the processes may be embodied within hardware, such as an application specific integrated circuit (“ASIC”) or otherwise.

A tangible machine-readable storage medium includes any mechanism that provides (i.e., stores) information in a non-transitory form accessible by a machine (e.g., a computer, network device, personal digital assistant, manufacturing tool, any device with a set of one or more processors, etc.). For example, a machine-readable storage medium includes recordable/non-recordable media (e.g., read only memory (ROM), random access memory (RAM), magnetic disk storage media, optical storage media, flash memory devices, etc.).

The above description of illustrated embodiments of the invention, including what is described in the Abstract, is not intended to be exhaustive or to limit the invention to the precise forms disclosed. While specific embodiments of, and examples for, the invention are described herein for illustrative purposes, various modifications are possible within the scope of the invention, as those skilled in the relevant art will recognize.

These modifications can be made to the invention in light of the above detailed description. The terms used in the following claims should not be construed to limit the invention to the specific embodiments disclosed in the specification. Rather, the scope of the invention is to be determined entirely by the following claims, which are to be construed in accordance with established doctrines of claim interpretation.

What is claimed is:

1. A rotary encoder, comprising:
a substrate;
one or more switches disposed on the substrate;
a mechanical wave generator disposed proximate to the substrate, wherein the substrate and the mechanical wave generator are adapted to rotate relative to each other about a central axis, the mechanical wave generator having a profile shape that repeats angularly about the central axis, the profile shape having ridges and valleys that engage the one or more switches to activate and deactivate the one or more switches as the substrate and the mechanical wave generator rotate relative to each other; and
a controller electrically coupled to the one or more switches to track activations of the one or more switches and digitally encode a rotational position of the substrate relative to the wave generator based upon the activations.

2. The rotary encoder of claim 1, wherein two or more of the switches are co-radially aligned on the substrate.

3. The rotary encoder of claim 1, wherein two or more of the switches are angularly offset from each other on the substrate such that only a single one of the two or more switches are activated by the ridges at a given time,

4. The rotary encoder of claim 1, wherein each of the switches corresponds to an encoding channel of the rotary encoder and a total number of encoding states per revolution between the substrate and the mechanical wave generator comprises twice a total number of the ridges multiplied by a total number of the switches.

5. The rotary encoder of claim 1, wherein the one or more switches are electro-mechanical switches each comprising:
a contact pad disposed on a surface of the substrate; and
a flexible contact lead having a first end and a second end, the first end mounted to the substrate and the second end cantilevered over the contact pad, wherein the second end of the flexible contact lead is reciprocally pushed against the contact pad by the ridges of the mechanical wave generator to form a closed circuit as the mechanical wave generator and the substrate rotate relative to each other.

6. The rotary encoder of claim 1, wherein the ridges of the mechanical wave generator are fabricated of an electrically insulating material.

7. The rotary encoder of claim 1, wherein the substrate is stationary and the mechanical wave generator is mounted to rotate.

8. The rotary encoder of claim 7, wherein the mechanical wave generator further comprises:
a ring substrate having the profile shape with the ridges and the valleys formed in the ring substrate.

9. The rotary encode of claim 8, wherein the mechanical wave generator further comprises:
a shaft aligned with the central axis; and
a platform connected to the shaft, wherein the ring substrate is disposed on the platform.

10. The rotary encoder of claim 8, wherein the profile shape that repeats angularly about the central axis comprises one of:
a triangular shape;
a truncated triangular shape;
an undulating shape; or
a trapezoidal shape.

11. The rotary encoder of claim 1, wherein the controller is disposed on the substrate.

12. The rotary encoder of claim 1, wherein the rotary encoder is disposed within a drug injection pen and one of the substrate or the mechanical wave generator is coupled to rotate with a drug injection mechanism of the drug injection pen such that a relative rotational movement between the substrate and the mechanical wave generator correlates to a volume of fluid dispensed from the drug injection pen.

13. A method of tracking a dosage of a fluid dispensed from a drug injection pen, comprising:
receiving a rotary motion at a rotary encoder disposed at least in part in a button attached to an end of the drug injection pen, the rotary motion received from a dosage injection mechanism or a dosage selection mechanism of the drug injection pen;
rotating a substrate of the rotary encoder within the button relative to a mechanical wave generator of the rotary encoder, the rotating based upon the rotary motion received from the dosage injection mechanism or the dosage selection mechanism;
reciprocally activating and deactivating one or more switches disposed on the substrate as the substrate rotates relative to the mechanical wave generator, wherein each of the one or more switches are reciprocally activated and deactivated in response to engagement between the mechanical wave generator and the one or more switches;
encoding the activations or the deactivations, or both, of the one or more switches with a controller coupled to the one or more switches; and
generating a signal with the controller based upon the encoding, wherein the signal is indicative of the dosage of the fluid dispensed over a period of time.

14. The method of claim 13, wherein the switches and the controller are both disposed on the substrate.

15. The method of claim 13, wherein reciprocally activating and deactivating the one or more switches comprises:
reciprocally pressing a flexible contact lead mounted to the substrate against a contact pad disposed on the substrate by reciprocally pressing ridges of the mechanical wave generator against the flexible contact lead.

16. The method of claim 15, wherein the mechanical wave generator comprises a ring substrate having a profile shape with the ridges and valleys formed in the ring substrate.

17. The method of claim 16, wherein the ridges of the mechanical wave generator are formed from an electrically insulating material.

18. The method of claim 13, wherein reciprocally activating and deactivating the one or more switches comprises:
reciprocally activating and deactivating two or more switches on the substrate with the mechanical wave generator.

19. The method of claim 18, wherein each of the switches corresponds to an encoding channel of the rotary encoder and a total number of encoding states per revolution between the substrate and the mechanical wave generator comprises twice a total number of ridges on the mechanical wave generator multiplied by a total number of the switches.

20. The method of claim 13, wherein the rotary encoder is disposed within a pen button that attaches to a distal end of the drug injection pen.
